(19) United States
(12) Patent Application Publication
(10) Pub. No.: US 2025/0127565 A1
(43) Pub. Date: Apr. 24, 2025

(54) ELECTROSURGICAL TRANSSEPTAL ASSEMBLY WITH MULTIPURPOSE CONTROL

(71) Applicant: Boston Scientific Medical Device Limited, Galway (IE)

(72) Inventors: Lauren Koon, Mississauga (CA); Gareth Davies, Toronto (CA); Jackie Leung, Richmond Hill (CA)

(21) Appl. No.: 18/920,475

(22) Filed: Oct. 18, 2024

Related U.S. Application Data
(60) Provisional application No. 63/591,815, filed on Oct. 20, 2023.

(51) Int. Cl. A61B 18/14; A61B 18/00; A61B 18/12

(52) U.S. Cl. CPC A61B 18/1492; A61B 18/1206; A61B 2018/00178; A61B 2018/00184; A61B 2018/00351; A61B 2018/00702; A61B 2018/0091; A61B 2018/1425; A61B 2018/1475

(57) ABSTRACT

An electrosurgical device for use with a radiofrequency (RF) energy source to provide RF energy is disclosed. The electrosurgical device includes a delivery component having an elongate shaft having a distal portion and a proximal portion and forming a lumen. The distal portion includes a delivery component distal end. A crossing member is disposed within the lumen. The crossing member includes a crossing member distal tip extendable from the delivery component distal end to deliver the RF energy from the RF energy source. A handle is coupled to the proximal portion of the shaft. An actuator is coupled to the handle. The actuator extends the crossing member distal tip from the delivery component distal end and couples the RF energy source to the crossing member such that RF energy is delivered to the distal tip of the crossing member.

### CROSS REFERENCE TO RELATED APPLICATIONS

This application claims priority to U.S. Provisional Application No. 63/591,815 entitled “ELECTROSURGICAL TRANSSEPTAL ASSEMBLY WITH MULTIPURPOSE CONTROL,” filed Oct. 20, 2023, which is hereby incorporated by reference in its entirety.

### TECHNICAL FIELD

The present disclosure relates to medical devices and systems for use in percutaneous or interventional procedures including surgery. More specifically, this disclosure relates to electrosurgical devices, assemblies, and systems that provide for cutting or puncturing of bodily tissues with an electrode.

### BACKGROUND

Catheters are often used to provide general access into a patient's body using minimally invasive techniques. In some examples, a catheter can be used to create a channel through a region of the body. One such example is a transseptal puncture in a cardiac procedure. The left atrium is a difficult cardiac chamber to access reach percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools. The transseptal puncture permits a direct route to the left atrium via the intra-atrial septum and venous system. The use of this technique permits for the introduction of larger and more complex medical devices into the left atrium. Historically, the technique was used exceptionally for mitral valvuloplasty and ablation in the left heart. Today, the increased interest in catheter ablation and its application in many other procedures has meant the transseptal puncture is a routine technique for interventional cardiologists and cardiac electrophysiologists.

Transseptal punctures are now performed with the aid of guidewires having needles or electrodes energized with a suitable power source such as an electrically coupled power generator in a manner similar to electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver electrical energy to the area where tissue is to be affected. The electrosurgical devices are typically held by the surgeon and connected to the power source, such as the electrosurgical unit, via cabling.

Electrosurgical devices pass electrical energy through tissue between the electrodes to cut or puncture tissue with plasma formed on the energized electrode. Tissue that contacts the plasma experiences a rapid vaporization of cellular fluid to produce a cutting effect. Typically, cutting is performed with electrodes in the monopolar arrangement. Electrical energy can be applied to the electrodes either as a train of high frequency pulses or as a continuous signal typically in the radiofrequency (RF) range to perform the cutting or puncturing techniques.

### SUMMARY

In an Example 1, an electrosurgical device for use with a radiofrequency (RF) energy source to provide RF energy, the electrosurgical device comprising: a delivery component having an elongate shaft having a distal portion and a proximal portion and forming a lumen, the distal portion including a delivery component distal end; a crossing member disposed within the lumen, the crossing member having a crossing member distal tip extendable from the delivery component distal end and configured to deliver the RF energy from the RF energy source; a handle coupled to the proximal portion of the shaft; and an actuator coupled to the handle, the actuator configured to extend the crossing member distal tip from the delivery component distal end and couple the RF energy source to the crossing member, such that RF energy is delivered to the distal tip of the crossing member.

In an Example 2, the electrosurgical device of Example 1, wherein the electrosurgical device is coupled to an introducer to form a multipurpose transseptal assembly.

In an Example 3, the electrosurgical device of Example 2, wherein multipurpose transseptal assembly is coupled to an electrosurgical generator configured to provide the RF energy.

In an Example 4, the electrosurgical device of Example 3, wherein the actuator provides a signal to the electrosurgical generator to request the RF energy.

In an Example 5, the electrosurgical device of any of Examples 1 to 4, wherein the actuator includes an advancement mechanism configured to extend the crossing member distal tip and a time delay circuit configured to deliver the RF energy, wherein the activation of the actuator delivers the RF energy after a selected time delay after the crossing member distal tip is extended.

In an Example 6, the electrosurgical device of Example 5, wherein the advancement mechanism includes a retractor mechanism configured to retract the extended crossing member distal tip into the distal end.

In an Example 7, the electrosurgical device of any of Examples 1 to 4, wherein the actuator includes an advancement mechanism and an RF delivery circuit, wherein activation of the actuator into a first position extends the crossing member and wherein activation of the actuator into a second position from the first position delivers the RF energy to the extended crossing member.

In an Example 8, the electrosurgical device of any of Examples 1 to 4, wherein the actuator includes a first actuator coupled to an advancement mechanism configured to extend the crossing member and a second actuator is coupled to an RF delivery circuit configured to deliver the RF energy to the extended actuator.

In an Example 9, the electrosurgical device of Example 7, wherein the advancement mechanism is configured to lock the extended crossing member in place relative to the delivery component distal end.

In an Example 10, the electrosurgical device of any of Examples 1 to 8, wherein the actuator includes one of a pushbutton, slide mechanism, and trigger.

In an Example 11, the electrosurgical device of Example 10, wherein the actuator further includes a linkage coupled to the one of the pushbutton, slide mechanism, and trigger and to the crossing member.

In an Example 12, the electrosurgical device of any of Examples 1-11, wherein the delivery component is a dilator, and the delivery component distal end includes a dilator tip.

In an Example 13, the electrosurgical device of any of Examples 1-11, wherein the delivery component is a delivery sheath.

In an Example 14, the electrosurgical device of any of Examples 1-13, wherein the actuator includes one of a slide mechanism and a spring-loaded mechanism in the handle, the slide mechanism and spring-loaded mechanism configured to extend the crossing member.

In an Example 15, the electrosurgical device of Example 1, further including an auxiliary sheath having a lumen for accepting the delivery component.

In an Example 16, an electrosurgical device for use with a radiofrequency (RF) energy source to provide RF energy, the electrosurgical device comprising: a delivery component having an elongate shaft having a distal portion and a proximal portion and forming a lumen, the distal portion including a delivery component distal end; a crossing member disposed within the lumen, the crossing member having a crossing member distal tip extendable from the delivery component distal end and configured to deliver the RF energy from the RF energy source; a handle coupled to the proximal portion of the shaft; and an actuator coupled to the handle, the actuator configured to extend the crossing member distal tip from the delivery component distal end and couple the RF energy source to the crossing member, such that RF energy is delivered to the distal tip of the crossing member.

In an Example 17, the electrosurgical device of Example 16, wherein the actuator includes an advancement mechanism configured to extend the crossing member distal tip and a time delay circuit configured to deliver the RF energy, wherein the activation of the actuator delivers the RF energy after a selected time delay after the crossing member distal tip is extended.

In an Example 18, the electrosurgical device of Example 17, wherein the advancement mechanism includes a retractor mechanism configured to retract the extended crossing member distal tip into the distal end.

In an Example 19, the electrosurgical device of Example 16, wherein the actuator includes an advancement mechanism and an RF delivery circuit, wherein activation of the actuator into a first position extends the crossing member and wherein activation of the actuator into a second position from the first position delivers the RF energy to the extended crossing member.

In an Example 20, the electrosurgical device of Example 19, wherein the advancement mechanism is configured to lock the extended crossing member in place relative to the delivery component distal end.

In an Example 21, the electrosurgical device of Example 16, wherein the actuator includes one of a pushbutton, slide mechanism, and trigger.

In an Example 22, the electrosurgical device of Example 21, wherein the actuator further includes a linkage coupled to the one of the pushbutton, slide mechanism, and trigger and to the crossing member.

In an Example 23, the electrosurgical device of Example 16, wherein the actuator includes a first actuator coupled to an advancement mechanism configured to extend the crossing member and a second actuator is coupled to an RF delivery circuit configured to deliver the RF energy to the extended actuator.

In an Example 24, the electrosurgical device of Example 16, wherein the delivery component is a dilator, and the delivery component distal end includes a dilator tip.

In an Example 25, the electrosurgical device of Example 16, wherein the delivery component is a delivery sheath.

In an Example 26, the electrosurgical device of Example 16, wherein the actuator includes one of a slide mechanism and a spring-loaded mechanism in the handle, the slide mechanism and spring-loaded mechanism configured to extend the crossing member.

In an Example 27, the electrosurgical device of Example 16, further including an auxiliary sheath having a lumen for accepting the delivery component.

In an Example 28, an electrosurgical assembly for use with an electrosurgical generator. The electrosurgical assembly comprising: an introducer having an introducer handle and an elongate sheath forming an introducer lumen; and an integrated transseptal device. The integrated transseptal device comprising: an elongate shaft disposed within the introducer lumen, the elongate shaft having a distal end and a proximal end and forming a device lumen, the distal end including a dilator tip; a crossing member disposed within the device lumen, the crossing member having a distal tip extendable from the dilator tip and configured to apply a radiofrequency (RF) signal; a device handle coupled the introducer handle, the device handle attached to the shaft at the proximal end, and an actuator coupled to the device handle, the actuator configured to extend the distal tip of the crossing member from the dilator tip and deliver the RF signal to the extended crossing member.

In an Example 29, the electrosurgical assembly of Example 28, wherein the device handle is electrically coupled to the introducer handle.

In an Example 30, the electrosurgical assembly of Example 28, wherein the crossing member is hollow and receives a guidewire.

In an Example 31, the electrosurgical assembly of Example 28, wherein the actuator provides a signal to the electrosurgical generator to request RF energy.

In an Example 32, the electrosurgical assembly of Example 31, wherein the actuator includes an advancement mechanism configured to extend the crossing member distal tip and a time delay circuit configured to deliver the RF energy, wherein the activation of the actuator delivers the RF energy after a selected time delay after the crossing member distal tip is extended.

In an Example 33, an electrosurgical system comprising: an electrosurgical generator configured to provide a source of radiofrequency (RF) energy as an RF signal; and an electrosurgical assembly for use with an electrosurgical generator. The electrosurgical assembly comprising: an introducer having an introducer handle and an elongate sheath forming an introducer lumen; and an integrated transseptal device. The integrated transseptal device comprising: an elongate shaft disposed within the introducer lumen, the elongate shaft having a distal end and a proximal end and forming a device lumen, the distal end including a dilator tip; a crossing member disposed within the device lumen, the crossing member having a distal tip extendable from the dilator tip and configured to apply a radiofrequency (RF) signal; a device handle coupled the introducer handle, the device handle attached to the shaft at the proximal end, and an actuator coupled to the device handle, the actuator configured to extend the distal tip of the crossing member from the dilator tip and deliver the RF signal to the extended crossing member.

In an Example 34, the electrosurgical system of Example 33, wherein the device handle is electrically coupled to the introducer handle.

In an Example 35, the electrosurgical system of Example 33, wherein the actuator provides a signal to the electrosurgical generator to request the RF energy.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram illustrating an example system for treating a patient, such as a heart or the vasculature of a patient, including an electrosurgical generator and a multipurpose transseptal assembly, the multipurpose transseptal assembly including an introducer and an integrated transseptal device.

FIG. 2 is a schematic diagram illustrating an embodiment of an integrated transseptal device for use in the multipurpose transseptal assembly of FIG. 1.

FIG. 3 is a schematic diagram illustrating another embodiment of an integrated transseptal device for use in the multipurpose transseptal assembly of FIG. 1.

FIG. 4 is a schematic diagram illustrating another embodiment of an integrated transseptal device for use in the multipurpose transseptal assembly of FIG. 1.

FIGS. 5A and 5B are schematic diagrams illustrating an example embodiment of the integrated transseptal device of FIG. 2.

FIGS. 6A and 6B are schematic diagrams illustrating an example embodiment of a feature of the integrated transseptal device of FIG. 3.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

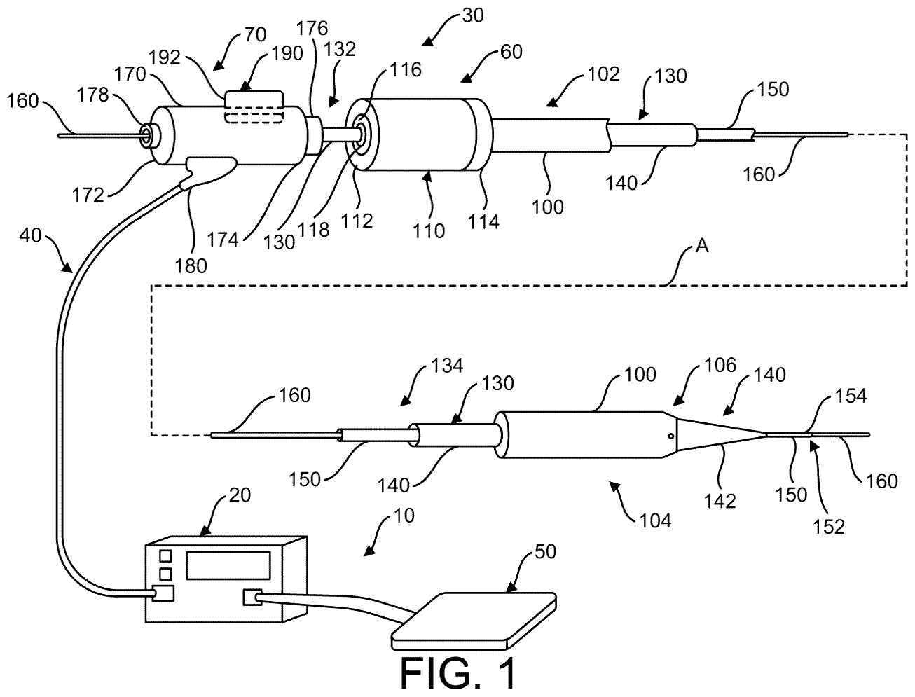

FIG. 1 illustrates an embodiment of a medical system 10 to facilitate vascular access to a heart and provide catheter positioning within cardiac anatomy. The embodiment of the medical system 10 includes an electrosurgical generator 20 and a crossing mechanism, such as a multipurpose transseptal assembly 30, electrically coupled to the electrosurgical generator 20 via an adapter cable 40. The electrosurgical generator 20 is configured to provide a source of energy, such as radiofrequency (RF) energy to the multipurpose transseptal assembly 30 via the electrical cable 40. In some embodiments, the electrical cable 40 is configured to carry electrical energy from the multipurpose transseptal assembly 30 to the electrical surgical generator 20 or another controller. In some embodiments, the system 10 includes a ground pad electrode, or indifferent (dispersive) patch electrode 50 for use with the multipurpose transseptal assembly 30 in a monopolar configuration. In some embodiments, the multipurpose transseptal assembly 30 is implemented in a bipolar configuration without an indifferent patch electrode. The multipurpose transseptal assembly 30 includes an introducer 60 that is configured to receive a delivery component, such as an integrated transseptal device 70. In the illustrated embodiment, the multipurpose transseptal assembly 30 also includes a guidewire 160 received within the integrated transseptal device 70. The system 10 can be implemented in conjunction with a visualization system such as a fluoroscopic visualization system employing a C-arm or an echocardiogram system such as a transesophageal electrocardiogram (TEE). In some embodiments, the system 10 can further include another adapter cable for coupling the multipurpose transseptal assembly 30 to an electrocardiogram (ECG) monitor to selectively measure heart activity from within the chambers of the heart.

The introducer 60 provides for percutaneous catheter introduction into the vasculature and into chambers of the heart. In some embodiments, the introducer 60 is a steerable introducer that provides for deflection or steering and facilitates catheter positioning. The introducer 60 includes an elongate introducer sheath 100 and an introducer handle 110. The introducer shaft 100 forms a longitudinally extending lumen along axis A and includes a proximal portion 102 and a distal portion 104 having a distal tip 106. In some embodiments, the introducer sheath 100 can include an inner lumen surface lined with a hydrophobic material such as polytetrafluoroethylene (PTFE) and an outer surface coated with a siloxane such as silicone. The distal portion 104 can include holes proximate the distal tip 106 to prevent cavitation of the introducer sheath 100 and a radiopaque marker such as one constructed from platinum at the distal tip 106 to aid in fluoroscopic visualization. The introducer handle 110 is configured to be grasped and manipulated by a user and includes a proximal end 112 and a distal end 114. The proximal portion 102 of the introducer sheath 100 is attached to the distal end 114 of the handle 110. The proximal end 112 includes a connector 116 for coupling to the integrated transseptal device 70. The proximal end 112 also includes a lumen 118 for receiving the integrated transseptal device 70. The handle 110 can include a steering mechanism (not shown), such as a knob or dial, that is used to deflect or steer introducer shaft 100. In some embodiments (not shown), the introducer handle 110 is fitted with a hemostasis valve to reduce blood loss during catheter introduction and a sideport with a stopcock to permit blood aspiration, fluid infusion, and pressure monitoring. In some embodiments, the introducer 60 provides a sheath for the delivery component. In some embodiments, the introducer is an axillary sheath for a delivery sheath.

The integrated transseptal device 70 in the illustrated embodiment combines a vessel dilator and transseptal crossing member into a single tool. The integrated transseptal device 70 includes an elongate device shaft assembly 130 having a proximal portion 132 coupled to a proximal handle 170. The device shaft assembly 130 is received within the proximal end 112 of the introducer handle 110 and the lumen of the introducer sheath 100 and includes a distal portion 134 that extends from the introducer sheath distal tip 106. The device shaft assembly 130 includes a dilator shaft 140 and a coaxial crossing member 150 received within a lumen of the dilator shaft 140. In some embodiments, the crossing member 150 is hollow and receives a guidewire 160 extending through the crossing member 150. The dilator shaft 140 and crossing member 150 track over the guidewire 160. The dilator shaft 140 includes a tapered dilator tip 142 on a distal portion. In some embodiments, the distal portion 104 of the introducer sheath 100 is also tapered to transition with the tapered dilator tip 142. The crossing member 150 includes a distal tip 152 extendable from the dilator tip 142. The distal tip 152 includes an exposed electrode 154. The crossing member 150 is configured to conduct an electrical signal, such as RF energy, from the proximal portion 132 of the shaft assembly 130 to the exposed electrode 154. The exposed electrode 154 is configured to apply RF energy to puncture tissue. In some embodiments, the crossing member 150 is a hollow, stainless-steel member within the dilator shaft 140. In some embodiments, the distal tip 154 of the crossing member 150 is configured in the form of a hollow needle to provide the option of mechanically puncturing tissue in addition to or instead of electrosurgically puncturing tissue.

The proximal handle 170 is configured to be grasped and manipulated by a user and includes a proximal end 172 and a distal end 174. The proximal portion 132 of the shaft assembly 130, including the proximal ends of the dilator shaft 140 and crossing member 150, is attached to the distal end 174 of the handle 170. The distal end 174 of the handle 170 includes a connector 176 for coupling to the connector 116 on the proximal end 112 of the introducer handle 110. The proximal end 172 of the proximal handle 170 in some embodiments includes a Luer-fitting guidewire lumen 178 to receive the guidewire 160 within the handle and to receive the guidewire 160 within the hollow crossing member 150. The proximal handle 170 also includes an electrical connector 180 that is configured to electrically couple the generator 20 to the crossing member 150 and provide RF energy to the crossing member 150.

The handle 170 includes a controller 190 to operate features of the integrated transseptal device 70. The controller 190 includes a manipulable actuator 192 on the proximal handle 170 coupled to a control mechanism 194 disposed within the handle 170. The actuator 192 is configured to be manipulated by the user to effect a function of the integrated transseptal device 70, such as to advance or retract the crossing member 150 or apply RF energy to the crossing member 150. The control mechanism 194 is configured to interface between the actuator and the crossing member 150 to apply the selected function. In some embodiments, the actuator 192 includes a pushbutton, touchpad, knob, slide button, dial, trigger, sensor, microphone, or toggle switch, disposed on the proximal handle 170. In some embodiments, the control mechanism 194 includes a linkage, a spring- or spring-loaded mechanism, magnet, gear, electrical switch, electrical circuit, or electromechanical device coupled to the actuator 192. In some embodiments, the actuator 192 can be depressed, slid, turned, twisted, toggled, touch, shifted, pulled, opened, closed, pushed to cause the control mechanism 194 to advance or retract the crossing member 150 or apply RF energy to the crossing member 150.

For instance, the device shaft assembly 130 is transitionable between a first configuration, in which the distal tip 152 and exposed electrode 154 of the crossing member 150 are disposed within the dilator shaft 140, such as the dilator tip 142, and a second configuration, in which the distal tip 152 and exposed electrode 154 of the crossing member 150 are extended from the distalmost portion of the dilator tip 142, as illustrated. The controller 190 can be used to transition the device shaft assembly between the first and second positions. In some embodiments, the actuator 192 is mechanically coupled to the crossing member 150 via control mechanism 194. The actuator 192 can be applied to cause the control mechanism 194 to advance or retract the distal tip 152 of the crossing member 150 with respect to the dilator tip 142. For instance, the controller 190 can include a slide mechanism coupled to the crossing member 150. The actuator 192 can be slid into a first position from which the slide mechanism will extend the distal tip 152 of the crossing member 150 from the dilator tip 142, and the actuator 192 can be slid into a second position from which the slide mechanism will retract the distal tip 152 of the crossing member 150 into the dilator tip 142. In another instance, the controller 190 can include a spring-tensioned linkage operated via a pushbutton actuator 192 to advance and retract the distal tip 152 of the crossing member 150 with respect to the dilator tip 142. Other configurations are contemplated. Additionally, the controller 190 can be used to selectively apply the RF energy to the electrode 154 on the crossing member 150. For example, the actuator 192 can include a pushbutton or trigger to activate a switch within the control mechanism 194 to close and allow the RF energy to pass from the generator 20 to the electrode 154 and open to prevent the RF energy to pass from the generator 20 the electrode 154. In another example, the actuator 192 can include a button to activate a circuit within control mechanism 194 to apply a signal to the generator 20 to request the RF energy that is applied to the electrode 154. Other configurations are contemplated. In some embodiments, the integrated transseptal device 70 or multipurpose transseptal assembly 30 can include a plurality of actuators, and in some embodiments an actuator 192 can be disposed on the introducer handle 110 and coupled to the mechanism 194 via the connectors 116, 176, which in some embodiments include an electrical coupling.

In an anticipated use of the system 10, the introducer 60 is attached to the integrated transseptal device 70 such that the shaft assembly 130 including the dilator shaft 140 and coaxial crossing member 150 are received within the introducer sheath 100. The introducer handle 110 is coupled to the proximal handle 170 at connectors 116, 176, and multipurpose transseptal assembly 30 is electrically coupled to the generator 20 via the electrical cable. If the multipurpose transseptal assembly 30 is to be configured in a monopolar mode, the patch electrode 50 is coupled to the patient. The generator 20 can be set at to a Cut mode, such as an energy output of approximately 10 watts. In some examples, femoral access is obtained via a conventional percutaneous needle, and the guidewire 160 is inserted into the vasculature and advanced to the superior vena cava. The proximal end of the guidewire 160 is inserted onto the shaft assembly 130, such as over the distal tip 152 of the hollow crossing member 150. The guidewire 160 exits the proximal handle 170 at the guidewire lumen 178. The introducer sheath 100 and shaft assembly 130 are advanced over the guidewire 160 to the superior vena cava. Under visualization, the dilator tip 142 is moved from the superior vena cava to the right atrial septum and then to the fossa ovalis of the heart. Once the dilator tip 142 is confirmed at the fossa ovalis, the electrode 154 of the crossing member 150 is advanced from the dilator tip 142 via the control mechanism 190. In one example, the distalmost tip of the crossing member 150 extends approximately four millimeters from the distal tip 142 when fully extended. The forward pressure is applied and the control mechanism 190 is actuated to apply the RF energy to the electrode 154 and puncture the fossa ovalis. The guidewire 160 can be advanced into the left atrium of the heart, and the crossing member 150 is retracted into the dilator tip 142. The dilator shaft 140 is advanced until the introducer sheath 100 is inside the left atrium. The proximal handle 170 detached from the introducer handle 110, and the shaft assembly 130 is removed from the patient.

Without ergonomic considerations to the design, the system 10 can be unwieldly to use. For instance, the controls on the multipurpose transseptal assembly 30 can be difficult to operate while a user is attempting to maintain pressure on or position of the dilator tip 142. Often, two hands are applied to the handles 110, 170 of the multipurpose transseptal assembly 30 to position the distal tip 142 and protrude the crossing member 150 with the control mechanism 190. In some instances, a clinician will employ an underhanded grip to better control rotational security of the dilator tip 142 and actuate the control mechanism 190 to protrude the crossing member 150 from the dilator tip 142.

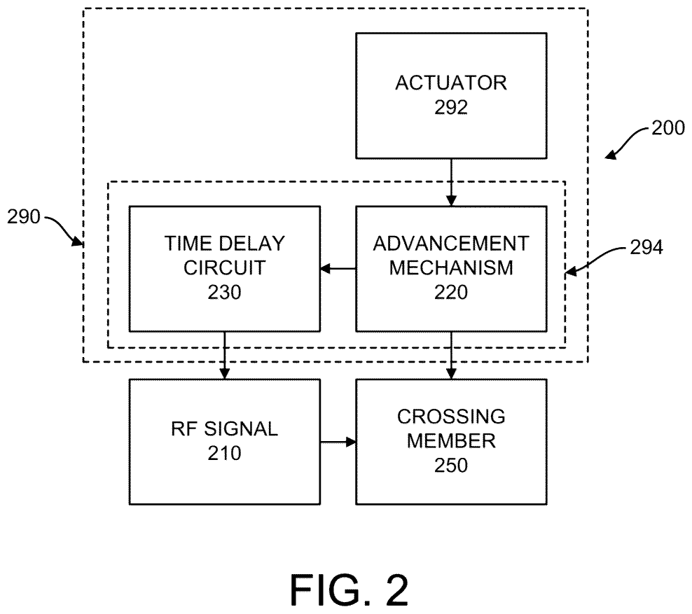

FIG. 2 illustrates a schematic diagram of an integrated transseptal device 200 such as a device corresponding with integrated transseptal device 70 implementing the controller 190 on the proximal handle 170 and in association with the crossing member 150. The integrated transseptal device 200 can be implemented having a shaft assembly including a positionable crossing member 250 coaxially disposed within the dilator in which the crossing member 250 includes a distal end electrode to apply an RF energy 210, the distal end electrode extendable from the dilator tip in a first position and retractable into the dilator tip in a second position. The integrated transseptal device 200 includes a proximal handle with a controller 290, corresponding with controller 190, to selectively advance and retract the crossing member 250 and to apply the RF energy 210 (received from a generator) via an actuator 292 in combination with a control mechanism 294.

In the illustrated embodiment, the actuator 292 is coupled to an advancement mechanism 220 of the control mechanism 294. The control mechanism 294 includes an advancement mechanism 220 configured to advance the distalmost tip and electrode of the crossing member 250 from the dilator tip in response to the actuator 292. In some embodiments, the advancement mechanism 220 can also retract the distalmost tip and electrode of crossing member 250 back into the dilator tip such as to include features of a retractor mechanism. In this embodiment, the actuator 292 can be manipulated to advance the crossing member 250 and manipulated again the retract the crossing member 250. In another embodiment, the actuator 292 can be manipulated and held in place to advance the crossing member 250 from the dilator tip, and the actuator 292 can be released to retract the crossing member 250 into the dilator tip. In some embodiments, the actuator 292 is a pushbutton, spring loaded trigger, or a slid button, and the advancement mechanism 220 is a linkage. The actuator 292 is coupled to a linkage to extend the crossing member 250. The control mechanism 290 also includes a time delay circuit 230 operable in response to the advancement mechanism 220. In addition to extending the crossing member 250 from the dilator tip, the advancement mechanism 220 activates the time delay circuit 230. The time delay circuit 230 is configured to delay a selected amount of time after activation, and then apply the RF energy 210 to the crossing member 250. In one embodiment, the time delay circuit 230 includes a switch that is closed after the selected amount of time to allow the RF energy 210 to pass to the crossing member 250. In another embodiment, the time delay circuit 230 provides a signal to the generator after the selected amount of time to request the generator provide the RF energy 230 to the integrated transseptal device 200. In this configuration, the RF energy 210 is not applied to the crossing member 250 until after the crossing member 250 is in the extended position. In one embodiment, the manipulation of the actuator 292 to retract the crossing member 250 can stop the RF energy to the crossing member 250 such as by opening a switch carrying the RF energy to the crossing member 250. In another embodiment, the RF energy can be on a timer and turn off after a selected amount of time for a burst. In another embodiment, the actuator 292 can be manipulated and held in place to advance the crossing member 250 from the dilator tip, wherein an RF energy is applied after the selected time delay, and the actuator 292 can be released to cut off or disconnect the RF energy and retract the crossing member into the dilator tip. In one embodiment, a single actuator 292 with a single manipulation can be used to separately advance the crossing member 250 from within the dilator tip and then apply RF energy 210 to the extended crossing member 250.

Integrated transseptal device 200 provides an embodiment of a single actuator 292 on the handle that is manipulated once, such as moved to first position or configuration, to provide a plurality of functions spaced-apart in time, namely a single button, slide, or trigger to be manipulated and extend the crossing member 250 and subsequently apply an RF energy 210 to the extended crossing member 250. The single actuator 292 can be manipulated again, such as moved back to a starting position or configuration, to retract the crossing member 250 into the dilator tip. The applied RF energy 210 can be cut off or disconnected prior to moving the manipulating the actuator 292 again or in connection with manipulating the actuator 292 again. The device 200 can include a feature to prevent the RF energy 210 from being applied to the crossing member 250 when the crossing member 250 is retracted in the dilator tip (which, in embodiments, includes prior to being advanced from the dilator tip) or if the crossing member 250 is advanced more than a predetermined distance, such as overextended from the dilator tip.

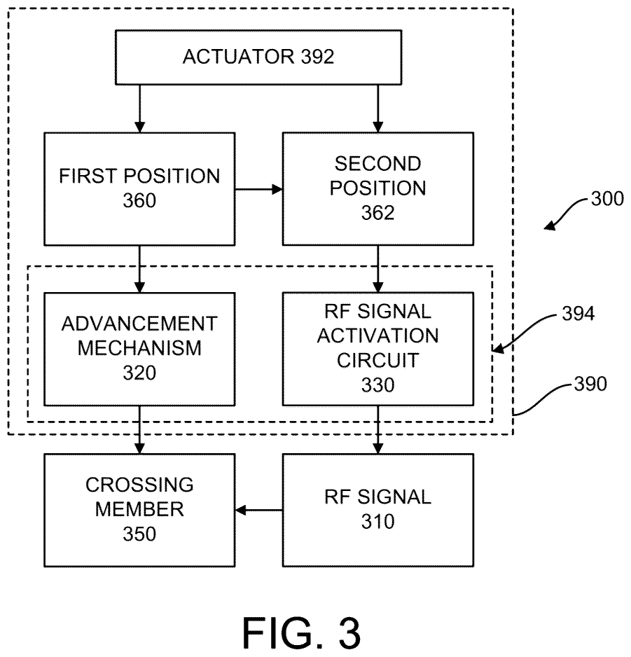

FIG. 3 illustrates a schematic diagram of an integrated transseptal device 300 such as a device corresponding with integrated transseptal device 70 implementing the controller 190 on the proximal handle 170 and in association with the crossing member 150. The integrated transseptal device 300 can be implemented having a shaft assembly including a positionable crossing member 350 coaxially disposed within the dilator in which the crossing member 350 includes a distal end electrode to apply an RF energy 310, the distal end electrode extendable from the dilator tip in a first configuration and retractable into the dilator tip in a second configuration. The integrated transseptal device 300 includes a proximal handle with a controller 390, corresponding with controller 190, to selectively advance and retract the crossing member 350 and to apply the RF energy 310 (received from a generator) via an actuator 392 in combination with a control mechanism 394.

In the illustrated embodiment, the actuator 392 is a multiple position device, such as a button, knob, trigger, or slide controller movable between a starting position and a plurality of active positions. For example, the actuator 392 is movable from a starting position to a first position. From the first position, the actuator 392 is movable to a second position, and in some embodiments back to the starting position. From the second position, the actuator 392 is moveable back to the starting position, and in some embodiments back to the first position. The actuator 392 is coupled to an advancement/retractor mechanism 320 and to an RF energy activation circuit 330. The advancement mechanism 320 configured to advance the distalmost tip and electrode of the crossing member 350 from the dilator tip in response to the actuator 392. The RF energy activation circuit 330 is configured to apply the RF energy 310 to the crossing member 350 in response to the actuator 392. In the illustrated embodiment, the actuator 392 is moved to a first position 360, such as from the starting position to the first position 360, which activates the advancement mechanism 320 to extend the crossing member 350 from the dilator tip. The actuator 392 is moved to the second position 362, such as from the first position 360 to the second position 362, which activates the RF energy activation circuit 330 to apply the RF energy 310 to the crossing member 350. In this configuration, the RF energy 310 is not applied to the crossing member 350 until after the crossing member 350 is in the extended position. Manipulation of the actuator 392 away from the second position 362, such as back to the starting position or to the first position can lock out the RF energy 310 from being applied to the crossing member 350. In one embodiment, the advancement mechanism 320 is a combination advancement and retractor mechanism that is configured to also retract the crossing member selectively 350 into the dilator tip. The actuator 392 can be returned to the starting position to retract the crossing member 350 to within the dilator tip and not provide RF energy 310 to the crossing member 350. In one embodiment, a single actuator 392 with multiple manipulations can be used to separately advance the crossing member 350 from within the dilator tip and then apply RF energy 310 to the extended crossing member 350.

Integrated transseptal device 300 provide an embodiment of a single actuator 392 on the handle that is manipulated twice, such as moved to a first position and then to a second position, to provide the plurality of functions spaced-apart in time, namely, a single button, slide, or trigger, to be manipulated to a first position to extend the crossing member 350, and then manipulated from the first position to a second position to apply the RF energy 310 to the crossing member 350. The single actuator 392 can be manipulated again, such as moved back to the first position from the second position, to cut off or disconnect the RF energy 310 from the crossing member 350, or such as moved back to a starting position or configuration, to retract the crossing member 350 into the dilator tip and cut off or disconnect the RF energy 310 from the crossing member 350. The device 300 can include a feature to prevent the RF energy 310 from being applied to the crossing member 350 when the crossing member 350 is retracted in the dilator tip (which, in embodiments, includes prior to being advanced from the dilator tip) or if the crossing member 350 is advanced more than a predetermined distance, such as overextended from the dilator tip.

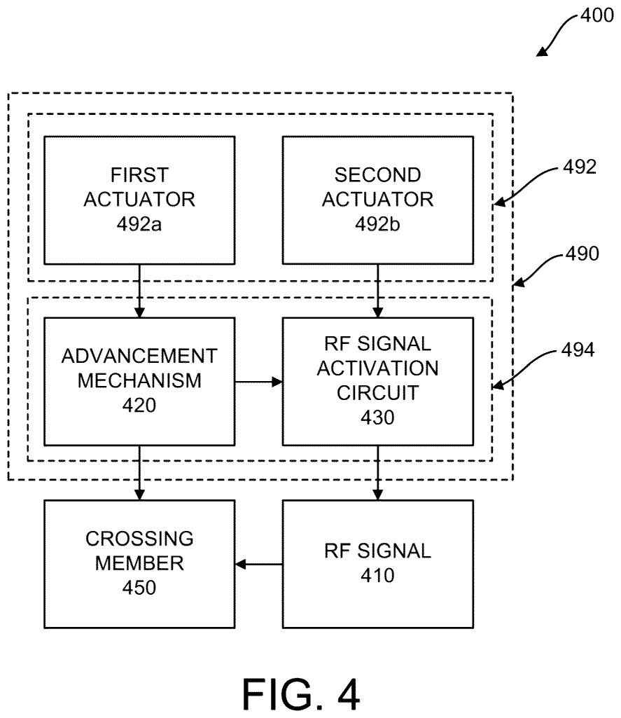

FIG. 4 illustrates a schematic diagram of an integrated transseptal device 400 such as a device corresponding with integrated transseptal device 70 implementing the controller 190 on the proximal handle 170 and in association with the crossing member 150. The integrated transseptal device 400 can be implemented having a shaft assembly including a positionable crossing member 450 coaxially disposed within the dilator in which the crossing member 450 includes a distal end electrode to apply an RF energy 410, the distal end electrode extendable from the dilator tip in a first position and retractable into the dilator tip in a second position. The integrated transseptal device 400 includes a proximal handle with a controller 490, corresponding with controller 190, to selectively advance and retract the crossing member 450 and to apply the RF energy 410 (received from a generator) via an actuator 492 in combination with a control mechanism 494.

In the illustrated embodiment, a first actuator 492a is coupled to an advancement mechanism 420 of the control mechanism, and a second actuator 492b is coupled to an RF energy activation circuit 430. The first actuator 492a and second actuator 492b each include a starting position and a single associated active position. The advancement mechanism 420 is configured to advance the distalmost tip and electrode of the crossing member 450 from the dilator tip in response to the first actuator 492a manipulated to the active position. In some embodiments, the advancement mechanism 420 can also retract the distalmost tip and electrode of crossing member 450 back into the dilator tip such as to include features of a retractor mechanism. The RF energy activation circuit 430 is configured to apply the RF energy 410 to the crossing member 450 in response to the second actuator 492b manipulated to the active position and either the first actuator 492a manipulated to the active position or the advancement mechanism 420 having advanced the distalmost tip and electrode of the crossing member 450 from the dilator tip. In one embodiment, the advancement mechanism 420 can have a lock mechanism to maintain the crossing member 450 in position once extended from the distal tip without further manipulation of the first actuator 492a. In this embodiment, the user can apply one finger or thumb to the first actuator 492a to extend the crossing member 450 and then use the same finger or thumb to manipulate the second actuator 492b to apply the RF energy signal 410.

In some embodiments, integrated transseptal device 400 is included as part of the multipurpose transseptal assembly, such as multipurpose transseptal assembly 30 wherein the second actuator 492b is disposed on the introducer handle 110. The second actuator 492b is electrically coupled to the RF energy activation circuit 430 in the integrated transseptal device and to the generator 20 via electrical connectors 116, 176. In some embodiments, both the second actuator 492b and the RF energy activation circuit 430 are included with the introducer handle 110 and are electrically coupled to the generator 20 via electrical connectors 116, 176.

Integrated transseptal device 400 provide an embodiment of multiple actuators 492a, 492b on the handle that are manipulated in succession, such as the first actuator 492a moved to an associated active position and then a second actuator moved to an associated active position, to provide the plurality of functions spaced-apart in time, namely, a two buttons, slides, triggers, or combinations of different actuators, in which the first actuator 492a is first manipulated, such as by a finger or thumb, to extend the crossing member 450, and then the second actuator 492b is manipulated, such as by the thumb or the same or another finger, to apply the RF energy 310 to the crossing member 350. The second actuator 492b can be released from the active position to not apply the RF energy 410 to the crossing member 450. The first actuator 492a can be manipulated again, such as moved back to the starting position from the active position, to retract the crossing member 450 into the dilator tip. The device 400 includes a feature to prevent the RF energy 410 from being applied to the crossing member 450 when the crossing member 450 is retracted in the dilator tip (which, in embodiments, includes prior to being advanced from the dilator tip) or if the crossing member 250 is advanced more than a predetermined distance, such as overextended from the dilator tip.

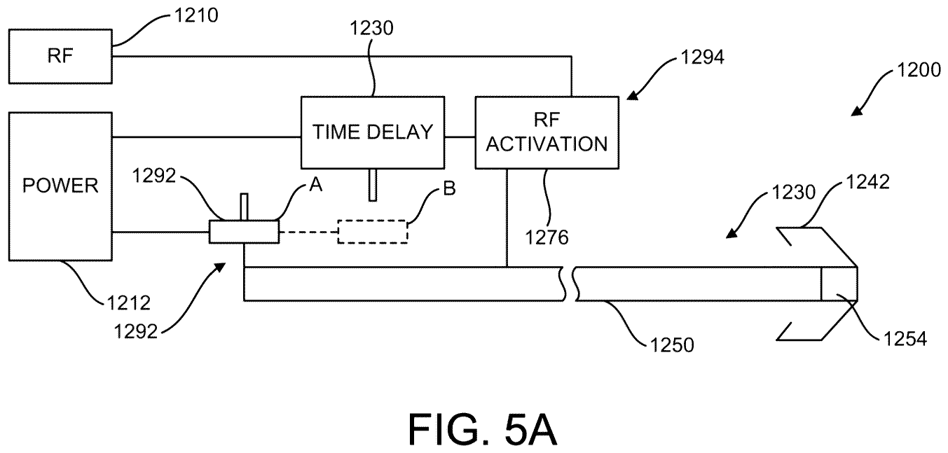

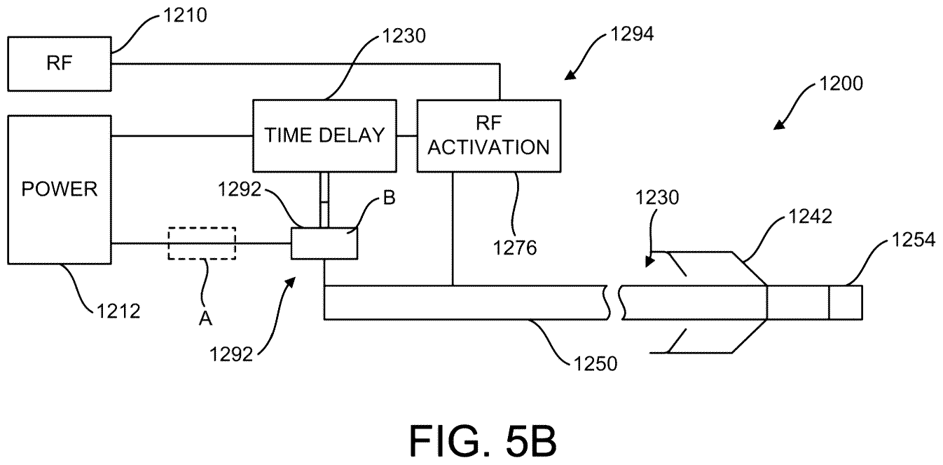

FIGS. 5A and 5B illustrate an example integrated transseptal device 1200 corresponding with the embodiment of the transseptal device illustrated in FIG. 2. FIG. 5A illustrates the transseptal device 1200 in a retracted, or first position. FIG. 5B illustrates the transseptal device 1200 in an extended, or second position. The integrated transseptal device 1200 can be implemented having a shaft assembly 1230 including a positionable crossing member 1250 coaxially disposed within the dilator 1240 in which the crossing member 1250 includes a distal end electrode 1254 to apply an RF energy 1210, the distal end electrode 1254 retractable into the dilator tip 1242 in a first position of FIG. 5A and extendable from the dilator tip 1242 in a second position of FIG. 5B. The integrated transseptal device 1200 includes a proximal handle with a controller 1290, corresponding with controller 290, to selectively advance and retract the crossing member 1250 and to apply the RF energy 1210 (received from a generator) via an actuator 1292 in combination with a control mechanism 1294.

In the illustrated embodiment, the actuator 1292 is coupled to an advancement mechanism 1220, such as a linkage, which is also coupled to the crossing member 1250. The advancement mechanism linkage 1220 is configured to advance the electrode 1254 of the crossing member 1250 from the dilator tip 1242 in response to the actuator 1292. The advancement mechanism linkage 1220 can also retract the electrode 1254 of crossing member 1250 back into the dilator tip 1242. In this embodiment, the actuator 1292 can be manipulated to advance the crossing member 1250 and manipulated again the retract the crossing member 1250. In another embodiment, the actuator 292 can be spring loaded so as to be manipulated and held in place to advance the crossing member 1250 from the dilator tip, and the actuator 1292 can be released to retract the crossing member 250 into the dilator tip. In the embodiment the actuator 1292 is a slide button movable from a first location A in the first position to a second location B in the second position. In FIG. 5A, the actuator 1292 is at first location A and second location B is shown in phantom. In FIG. 5B, the actuator 1292 is at second location B and first location A is shown in phantom.

The control mechanism 1290 also includes a time delay circuit 1230 and electrical coupling circuit 1276 operable in response to the actuator 1292. In addition to extending the crossing member 1250 from the dilator tip 1242, the actuator 1292 activates the time delay circuit 1230 in the second position. The time delay circuit 1230 is configured to delay a selected amount of time after activation, and then apply the RF energy 1210 to the crossing member 1250. In one embodiment, the time delay circuit 1230 includes a switch that is closed after the selected amount of time to allow the RF energy 1210 to pass to the crossing member 1250. The electrical coupling circuit 1276 includes circuitry and switches to couple the RF energy 1210 to the crossing member 1250 in response to the time delay circuit.

In the illustrated example, both the time delay circuit 1230 and the actuator 1292 are coupled to a source of electrical power 1212. The time delay circuit 1230 is coupled to a first pole 1272 of switch 1270 coupled to the electrical power 1212. The actuator 1292 is coupled to a second pole 1274 of switch 1212 coupled to electrical power 1212. As the slide button actuator 1292 is at location A in the first position of FIG. 5A, the poles 1272, 1274 of switch 1270 are open, and no power from electrical power 1212 runs through the actuator 1292 and time delay circuit 1230. As the slide button actuator 1292 is at location B in the second position of FIG. 5B, the poles 1272, 1274 of switch 1270 are together and closed, and power runs from electrical power 1212 through the actuator 1292 and time delay circuit 1230. The completed circuit of the actuator 1292 in location B activates the time delay circuit 1230, which, after a selected period of time, passes the RF energy 1210 to the crossing member via electrical coupling circuit 1276. another embodiment, the time delay circuit 230 provides a signal to the generator via electrical coupling circuit 1276 after the selected amount of time to request the generator provide the RF energy 1210 to the integrated transseptal device 1200. In this configuration, the RF energy 1210 is not applied to the crossing member 1250 until after the crossing member 1250 is in the extended position. In one embodiment, the manipulation of the actuator 292 to retract the crossing member 250 can stop the RF energy to the crossing member 1250 such as by opening a switch carrying the RF energy to the crossing member 1250 (not shown). In another embodiment, the RF energy can be on a timer such as via electrical coupling circuit 1276 and turn off after a selected amount of time for a burst. In the illustrated embodiment, a single actuator 1292 with a single manipulation can be used to separately advance the crossing member 1250 from within the dilator tip and then apply RF energy 1210 to the extended crossing member 1250.

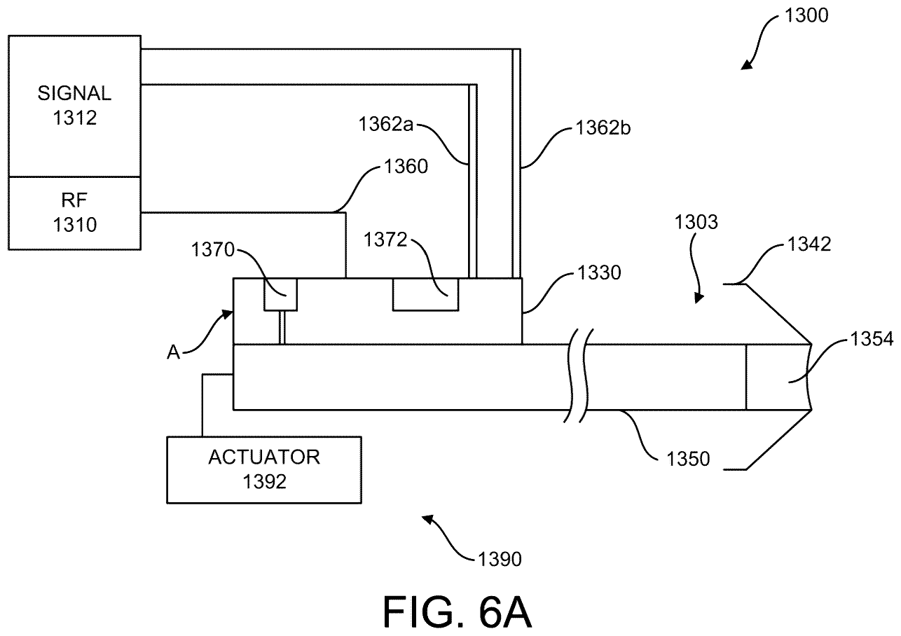

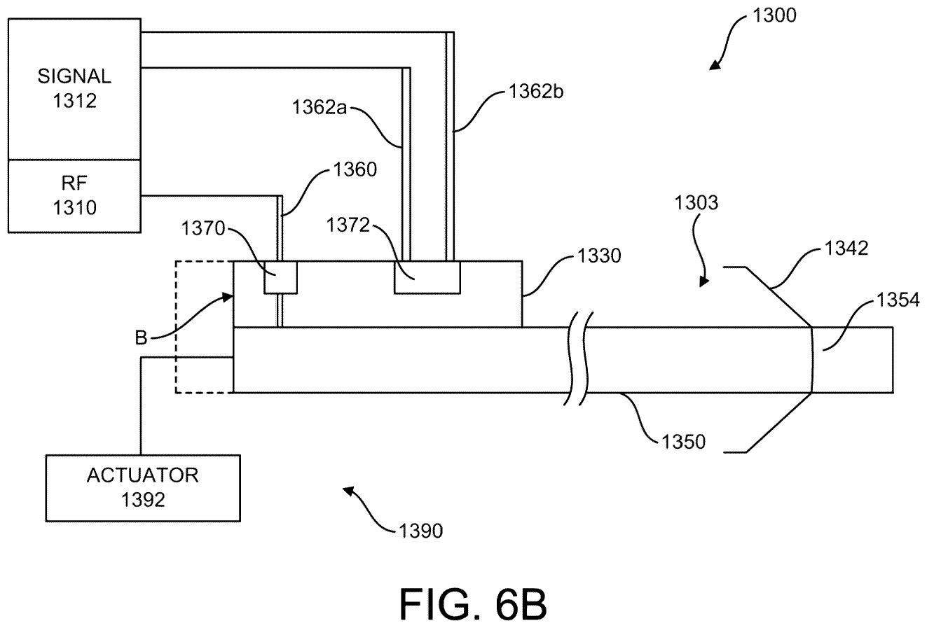

FIGS. 6A and 6B illustrate an example integrated transseptal device 1300 corresponding with the embodiment of the transseptal device to indicate an example interaction between the advancement mechanism 320, actuator 392, RF actuation circuit 330, crossing member 350, and RF signal 310 as illustrated, for example, in FIGS. 3 and 4. FIG. 6A illustrates the transseptal device 1300 in a retracted position. FIG. 6B illustrates the transseptal device 1300 in an extended position. The integrated transseptal device 1300 can be implemented having a shaft assembly 1303 including a positionable crossing member 1350 coaxially disposed within the dilator 1340 in which the crossing member 1350 includes a distal end electrode 1354 to apply an RF energy 1310, the distal end electrode 1354 retractable into the dilator tip 1342 in a retracted position of FIG. 6A and extendable from the dilator tip 1342 in an extended position of FIG. 6B. The integrated transseptal device 1300 includes a proximal handle with a controller 1390, corresponding with controller 290, to selectively advance and retract the crossing member 1350 and to apply the RF energy 1310 (received from a generator) via an actuator 1392 in combination with a control mechanism 1394.

In the illustrated embodiment, the actuator 1392 is coupled to an advancement mechanism 1320, such as a linkage, which is also coupled to the crossing member 1350 and the activation circuit 1330. The advancement mechanism linkage 1320 is configured to advance the electrode 1354 of the crossing member 1350 from the dilator tip 1342 in response to the actuator 1392. The advancement mechanism linkage 1320 can also retract the electrode 1354 of crossing member 1350 back into the dilator tip 1342. The advancement mechanism linkage is coupled to move, such as advance and retract, the activation circuit 1330. In this embodiment, the actuator 1392 can be manipulated to advance the crossing member 1350 and the activation circuit 1330 and manipulated again the retract the crossing member 1350 and the activation circuit 1330. In another embodiment, the actuator 392 can be spring loaded so as to be manipulated and held in place to advance the crossing member 1350 from the dilator tip, and the actuator 1392 can be released to retract the crossing member 350 into the dilator tip. In the embodiment the actuator 1392 is a slide button the can move the activation circuit 1330 from a first location A in the retracted position to a second location B in the extended position. In FIG. 6A, the activation circuit 1330 is at first location A. In FIG. 6B, the activation circuit is at second location B.

In the illustrated embodiment, the activation circuit 1330 is coupled to a source of RF energy 1310, via pathway, 1360 and to an electrical signal 1312, via pathway 1362a and 1362b, both from generator 20. The activation circuit 1330 includes an RF signal contact 1370 and an electrical signal contact 1372. The RF signal contact 1370 is coupled to the crossing member 1350. As the activation circuit 1330 is at location A, in FIG. 6A, the connections between the RF energy 1310 and crossing member 1350 are open and no RF energy is transferred to the crossing member 1350. Similarly, as the activation circuit 1330 is at location A, the connections between pathways 1362a and 1362b are open, as is electrical signal 1312. As the activation circuit 1330 is at location B, in FIG. 6B, the connections between the RF energy 1310 and crossing member 1350 are closed via contact 1370 and an RF energy can be transferred to the crossing member 1350 if provided at RF energy 1310. Similarly, as the activation circuit 1330 is at location B, the connections between pathways 1362a and 1362b are closed via contact 1372, and electrical signal 1312 is triggered at generator to provide the RF energy 1310.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electrosurgical device for use with a radiofrequency (RF) energy source to provide RF energy, the electrosurgical device comprising:
a delivery component having an elongate shaft having a distal portion and a proximal portion and forming a lumen, the distal portion including a delivery component distal end;
a crossing member disposed within the lumen, the crossing member having a crossing member distal tip extendable from the delivery component distal end and configured to deliver the RF energy from the RF energy source;
a handle coupled to the proximal portion of the shaft; and
an actuator coupled to the handle, the actuator configured to extend the crossing member distal tip from the delivery component distal end and couple the RF energy source to the crossing member, such that RF energy is delivered to the distal tip of the crossing member.

2. The electrosurgical device of claim 1, wherein the actuator includes an advancement mechanism configured to extend the crossing member distal tip and a time delay circuit configured to deliver the RF energy, wherein the activation of the actuator delivers the RF energy after a selected time delay after the crossing member distal tip is extended.

3. The electrosurgical device of claim 2, wherein the advancement mechanism includes a retractor mechanism configured to retract the extended crossing member distal tip into the distal end.

4. The electrosurgical device of claim 1, wherein the actuator includes an advancement mechanism and an RF delivery circuit, wherein activation of the actuator into a first position extends the crossing member and wherein activation of the actuator into a second position from the first position delivers the RF energy to the extended crossing member.

5. The electrosurgical device of claim 4, wherein the advancement mechanism is configured to lock the extended crossing member in place relative to the delivery component distal end.

6. The electrosurgical device of claim 1, wherein the actuator includes one of a pushbutton, slide mechanism, and trigger.

7. The electrosurgical device of claim 6, wherein the actuator further includes a linkage coupled to the one of the pushbutton, slide mechanism, and trigger and to the crossing member.

8. The electrosurgical device of claim 1, wherein the actuator includes a first actuator coupled to an advancement mechanism configured to extend the crossing member and a second actuator is coupled to an RF delivery circuit configured to deliver the RF energy to the extended actuator.

9. The electrosurgical device of claim 1, wherein the delivery component is a dilator, and the delivery component distal end includes a dilator tip.

10. The electrosurgical device of claim 1, wherein the delivery component is a delivery sheath.

11. The electrosurgical device of claim 1, wherein the actuator includes one of a slide mechanism and a spring-loaded mechanism in the handle, the slide mechanism and spring-loaded mechanism configured to extend the crossing member.

12. The electrosurgical device of claim 1, further including an auxiliary sheath having a lumen for accepting the delivery component.

13. An electrosurgical assembly for use with an electrosurgical generator, the electrosurgical assembly comprising:
an introducer having an introducer handle and an elongate sheath forming an introducer lumen; and
an integrated transseptal device, comprising:
an elongate shaft disposed within the introducer lumen, the elongate shaft having a distal end and a proximal end and forming a device lumen, the distal end including a dilator tip,
a crossing member disposed within the device lumen, the crossing member having a distal tip extendable from the dilator tip and configured to apply a radiofrequency (RF) signal,
a device handle coupled the introducer handle, the device handle attached to the shaft at the proximal end, and
an actuator coupled to the device handle, the actuator configured to extend the distal tip of the crossing member from the dilator tip and deliver the RF signal to the extended crossing member.

14. The electrosurgical assembly of claim 13, wherein the device handle is electrically coupled to the introducer handle.

15. The electrosurgical assembly of claim 13, wherein the crossing member is hollow and receives a guidewire.

16. The electrosurgical assembly of claim 13, wherein the actuator provides a signal to the electrosurgical generator to request RF energy.

17. The electrosurgical assembly of claim 16, wherein the actuator includes an advancement mechanism configured to extend the crossing member distal tip and a time delay circuit configured to deliver the RF energy, wherein the activation of the actuator delivers the RF energy after a selected time delay after the crossing member distal tip is extended.

18. An electrosurgical system comprising:
an electrosurgical generator configured to provide a source of radiofrequency (RF) energy as an RF signal; and
an electrosurgical assembly for use with an electrosurgical generator, the electrosurgical assembly comprising:
an introducer having an introducer handle and an elongate sheath forming an introducer lumen; and
an integrated transseptal device, comprising;
an elongate shaft disposed within the introducer lumen, the elongate shaft having a distal end and a proximal end and forming a device lumen, the distal end including a dilator tip,
a crossing member disposed within the device lumen, the crossing member having a distal tip extendable from the dilator tip and configured to apply the RF signal,
a device handle coupled the introducer handle, the device handle attached to the shaft at the proximal end, and
an actuator coupled to the device handle, the actuator configured to extend the distal tip of the crossing member from the dilator tip and deliver the RF signal to the extended crossing member.

19. The electrosurgical system of claim 18, wherein the device handle is electrically coupled to the introducer handle.

20. The electrosurgical system of claim 18, wherein the actuator provides a signal to the electrosurgical generator to request the RF energy.
